(19) United States
(12) Patent Application Publication
(10) Pub. No.: US 2024/0245709 A1
(43) Pub. Date: Jul. 25, 2024

(54) GANAXOLONE FOR USE IN TREATMENT OF ESTABLISHED STATUS EPILEPTICUS

(71) Applicant: MARINUS PHARMACEUTICALS, INC., Radnor, PA (US)

(72) Inventors: Henrikas VAITKEVICIUS, Radnor, PA (US); Maciej Gasior, Radnor, PA (US); Joseph Hulihan, Radnor, PA (US)

(21) Appl. No.: 18/625,718

(22) Filed: Apr. 3, 2024

Related U.S. Application Data
(63) Continuation of application No. PCT/US22/77373, filed on Sep. 30, 2022.
(60) Provisional application No. 63/252,090, filed on Oct. 4, 2021.

(51) Int. Cl. A61K 31/57; A61K 9/00; A61K 31/724

(52) U.S. Cl. CPC A61K 31/57; A61K 9/0019; A61K 31/724

(57) ABSTRACT

This invention relates to methods for treating established status epilepticus (ESE) by administering to the subject in need thereof an intravenous bolus of ganaxolone and a continuous intravenous infusion of a neurosteroid. The method provides ESE suppression and continued suppression of ESE.

The present application claims the benefit of U.S. Provisional Application No. 63/252,090, filed on Oct. 4, 2021, which is hereby incorporated by reference in its entirety.

### 1. BACKGROUND

Status epilepticus (“SE”) is a life-threatening neurological emergency associated with significant morbidity and mortality (Betjemann and Lowenstein, (2015) The Lancet Neurology, 14(6):615-624). In fact, it is the second most common neurologic emergency in the United States with approximately 150,000 cases per year and 55,000 associated deaths per year (Moghasddasi et al., (2015) J. Epilepsy Res., 5(1):13-16).

SE is manifested by prolonged seizure activity, typically persisting more than 5 minutes, or recurrent seizures without recovery of consciousness between seizures. Id. SE requires aggressive treatment to stop the seizure and prevent neurological damage, including neuronal death. SE becomes more difficult to control as its duration increases, and prolonged SE and refractoriness to treatment are associated with poor prognosis (Cherian & Thomas (2009), Ann. Indian. Acad. Neurol., 12(3): 140-153). Goals of treatment are rapid seizure cessation, maintenance of seizure control, preventing progression to anesthetics and avoiding further medical complications. Id.

Current treatment protocols for SE take a three-stage approach (Shorvon and Ferlisi (2011), Brain, 134(10)-2802-2818). The first-line treatment is typically with benzodiazepines (e.g., diazepam, lorazepam, and midazolam) (Trinka and Kälviäinen (2017), Seizure, 44:65-73: Glauser et al., (2016), Epilepsy Curr. 26(1):48-61). However, benzodiazepines are ineffective in about 35%-45% of cases and are associated with cardiovascular and respiratory side effects. Id. In instances when SE continues after first-line treatment, the patient is considered to have established SE (ESE). Ongoing seizure activity can lead to escalation of medications in an attempt to suppress and control seizure activity. As such, the subject can receive an excessive amount of potent medications (e.g., benzodiazepines, such as diazepam, lorazepam, and midazolam) and other anti-seizure medications (e.g., fosphenytoin, levetiracetam, and valproate), which can take months to wean the patient off. Moreover, ESE is linked to ongoing seizure activity, which may lead to significant complications and mortality, both from the ongoing seizures themselves and from the negative consequences of their treatment.

This disclosure relates to methods for treating ESE. Ganaxolone has been disclosed as being suitable for treating SE, particularly refractory SE by administering ganaxolone as an intravenous bolus plus a continuous intravenous infusion in an amount to maintain a plasma concentration of ganaxolone of about 400 ng/ml or higher for a target concentration period of at least about 8 hours or at least about 12 hours, as described in International Application No.: PCT/US2020/04484. The inventors have now discovered that a shorter treatment period, as brief as about 2 hours, can result in rapid and continued suppression of ESE. The new treatment regimen disclosed herein can prevent subjects having ESE progressing to a more severe form of SE, such as refractory SE or super refractory SE that is associated with high morbidity and mortality.

The methods disclosed herein comprise administering to a subject in need thereof a therapeutically effective course of a neurosteroid, preferably ganaxolone, as an intravenous bolus and a continuous intravenous infusion. The effective course of neurosteroid, preferably ganaxolone, can be administered in conjunction with the initial second-line AED. The intravenous bolus is administered in an amount to suppress SE. Suppression of SE reduces seizure burden. The continuous intravenous infusion is administered in an amount to prevent seizure recurrence. The effective course of neurosteroid, preferably ganavolone can suppress SE in a shorter duration relative to second-line AED. The effective course of neurosteroid, preferably ganaxolone can also result in persistent and durable cessation of SE compared to second-line AED.

Treatment with the course of neurosteroid (e.g., ganaxolone) typically last about 2 hours to about 10 hours, (e.g., about 2 hours, about 3 hours, about 4 hours, about 5 hours, about 6 hours, about 7 hours, about 8 hours, about 9 hours, about 10 hours) after which the continuous intravenous infusion is terminated. In preferred aspects, the course of neurosteroid (e.g., ganaxolone) lasts about 2 hours to about 4 hours. In preferred aspects the course of neurosteroid (e.g., ganaxolone) lasts about 2 hours. In preferred aspects, the course of neurosteroid (e.g., ganaxolone) lasts about 4 hours.

Treatment with the course of neurosteroid comprises an infusion of an amount of about 10 mg to about 30 mg of ganaxolone that is infused into the subject as an intravenous bolus. Preferably, about 20 mg of ganaxolone is infused into the subject as the intravenous bolus. The intravenous bolus can be administered (i.e., infused) into the subject for about 1 minute to about 5 minutes, preferably about 3 minutes.

During the continuous intravenous infusion about 40 mg to about 80 mg of ganaxolone per hour can be infused into the subject. Preferably about 60 mg of ganaxolone per hour is infused into the subject during the continuous intravenous infusion. The continuous intravenous infusion can be administered (i.e., infused) into the subject for about 2 hours to about 10 hours (e.g., about 2 hours, about 3 hours, about 4 hours, about 5 hours, about 6 hours, about 7 hours, about 8 hours, about 9 hours, about 10 hours) after which the continuous intravenous infusion is terminated.

The continuous intravenous infusion of ganaxolone is initiated periprocedural with the intravenous bolus. The continuous intravenous infusion is generally administered concurrently with the administration of the intravenous bolus. In some cases, the continuous intravenous infusion can be initiated before or after the intravenous bolus.

The methods disclosed herein can further comprise administering to a subject in need thereof one or more additional courses of medication with neurosteroid (preferably ganaxolone) administered as an intravenous bolus plus continuous intravenous infusion. Generally, if the subject shows signs of seizure re-lapses within about 12 hours from the initiation of a course of neurosteroid, the subject can be administered a second course of neurosteroid, typically for treatment period that is longer than the first course. For example if the first course treatment period was about 2 hours, the second course treatment period can be for about 4 hours to about 8 hours. Preferably, the second course treatment period is about 4 hours. If the subject shows signs of seizure relapse within about 30 minutes from completion of a course of neurosteroid, the second course of neurosteroid can optionally include administering to the subject an intravenous bolus. Typically, if the subject shows signs of seizure relapse within about 30 minutes from completion of a course of neurosteroid, the second course of neurosteroid comprises administering to the subject only the continuous infusion.

A third course of neurosteroid (e.g., ganaxolone), can be administered if the subject shows signs of seizure re-lapse within about 12 hours from the initiation from the second course of neurosteroid. Typically, the third course of neurosteroid is administered for a similar treatment period as the second course. For example, if the first course treatment period was about 2 hours and the second course treatment period was about 4 hours, the third treatment period can be for about 4 hours. Preferably, the third treatment period is about 4 hours. If the subject shows signs of seizure relapse within about 30 minutes from completion of a course of neurosteroid, the third course of neurosteroid can optionally include administering to the subject an intravenous bolus. Typically, if the subject shows signs of seizure relapse within about 30 minutes from completion of a course of neurosteroid, the third course of neurosteroid comprises administering to the subject only the continuous infusion.

The treatment course (i.e., the intravenous bolus plus continuous infusion) disclosed herein typically does not exceed 10 hours. Preferably, the first course and any additional courses of neurosteroid (e.g., ganaxolone) do not in the aggregate exceed a neurosteroid (e.g. ganaxolone) treatment period of about 10 hours. For example, the first course can be about 2 hours, the second course can be about 4 hours, and the third course can be about 4 hours. As another example, the first course can be about 2 hours and the second course can be about 8 hours.

Treatment with the one or more courses of neurosteroid can produce a plasma concentration in the subject of at least about 400 ng/ml to about 1000 ng/ml throughout the treatment period.

Accordingly, this disclosure relates to a method for effectively treating ESE that provides rapid suppression of ESE, sustained efficacy (i.e., prevents SE-relapse and provides for continued suppression of SE), and improved safety.

### 3. DETAILED DESCRIPTION

This disclosure relates to a new method for treating ESE. As described and exemplified herein, the method comprises administering to a subject in need thereof a therapeutically effective course of a neurosteroid, preferably ganaxolone, as an intravenous bolus and a continuous intravenous infusion. The effective course of neurosteroid is generally administered as adjuvant to the second line IV AED. The intravenous bolus is administered in an amount to suppress SE. Suppression of SE reduces seizure burden. The continuous intravenous infusion is administered in an amount for continued SE suppression. The method is used to treat subjects that have failed first-line treatment. In some practices of the method, escalation of additional medications is mitigated.

Treatment with the course of neurosteroid (e.g., ganaxolone) can be as brief as about 2 hours. Treatment with the course of neurosteroid (e.g., ganaxolone) can typically last about 2 hours to about 10 hours (e.g., about 2 hours, about 3 hours, about 4 hours, about 5 hours, about 6 hours, about 7 hours, about 8 hours, about 9 hours, about 10 hours) after which the continuous intravenous infusion is terminated. In preferred aspects, the course of neurosteroid (e.g., ganaxolone) lasts about 2 hours to about 4 hours. In preferred aspects the course of neurosteroid (e.g., ganaxolone) lasts about 2 hours. In preferred aspects, the course of neurosteroid (e.g., ganaxolone) lasts about 4 hours.

A treatment course of neurosteroid (e.g., ganaxolone) comprises administering to a subject in need thereof an intravenous bolus plus a continuous intravenous infusion. The intravenous bolus of neurosteroid (e.g., ganaxolone) is administered in an amount that is sufficient to suppress SE. The continuous intravenous bolus of neurosteroid (e.g., ganaxolone) is administered in an amount for continued suppression of SE. The intravenous bolus can be administered in an amount of about 10 mg to about 30 mg of ganaxolone. Preferably, about 20 mg of ganaxolone is infused into the subject as the intravenous bolus. The intravenous bolus can be administered (i.e., infused) into the subject for about 1 minute to about 5 minutes, preferably about 3 minutes.

During the continuous intravenous infusion about 40 mg to about 80 mg of ganaxolone per hour can be infused into the subject. Preferably about 60 mg of ganaxolone per hour is infused into the subject during the continuous intravenous infusion. The continuous intravenous infusion can be administered (i.e., infused) into the subject for about 2 hours to about 10 hours (e.g., about 2 hours, about 3 hours, about 4 hours, about 5 hours, about 6 hours, about 7 hours, about 8 hours, about 9 hours, about 10 hours) after which the continuous intravenous infusion is terminated.

The methods disclosed herein can further comprise administering to a subject in need thereof one or more additional courses of medication with neurosteroid (e.g., ganaxolone) administered as an intravenous bolus plus continuous intravenous infusions. Generally, if the subject shows signs of seizure re-lapses within about 12 hours from the initiation of a course of neurosteroid, the subject can be administered a second course of neurosteroid, typically for a treatment period that is longer than the first course. For example if the first course treatment period was about 2 hours, the second course treatment period can be for about 4 hours to about 8 hours. Preferably, the second course treatment period is about 4 hours. If the subject shows signs of seizure relapse within about 30 minutes after the completion of a course of neurosteroid, the second course of neurosteroid can optionally include administering to the subject an intravenous bolus. Typically, if the subject shows signs of seizure relapse within about 30 minutes from the 30 minutes after the completion of a course of neurosteroid, the second course of neurosteroid comprises administering a continuous infusion only (e.g., an intravenous is not administered).

A third course of neurosteroid (e.g., ganaxolone), can be administered if the subject shows signs of seizure re-lapse within about 12 hours from the initiation from the second course of neurosteroid. Typically, the third course of neurosteroid is administered for a similar treatment period as the second course. For example, if the first course treatment period was about 2 hours and the second course treatment period was about 4 hours, the third treatment period can be for about 4 hours. Preferably, the third treatment period is about 4 hours. If the subject shows signs of seizure relapse within about 30 minutes from the completion of a course of neurosteroid, the third course of neurosteroid can optionally include administering to the subject an intravenous bolus. Typically, if the subject shows signs of seizure relapse within about 30 minutes from completion of a course of neurosteroid, the third course of neurosteroid comprises administering to the subject a continuous infusion only.

The intravenous bolus of the one or more additional courses of medication with neurosteroid (e.g., ganaxolone) can be administered in the same amount as the first course. For instance, the intravenous bolus can be administered in an amount of about 10 mg to about 30 mg of ganaxolone. Preferably, about 20 mg of ganaxolone is infused into the subject as the intravenous bolus. The intravenous bolus can be administered (i.e., infused) into the subject for about 1 minute to about 5 minutes, preferably about 3 minutes. The continuous intravenous infusion of the one or more additional courses of medication with neurosteroid (e.g., ganaxolone) can be administered in the same amount as the first course. For instance, the continuous intravenous infusion about 40 mg to about 80 mg of ganaxolone per hour can be infused into the subject. Preferably about 60 mg of ganaxolone per hour is infused into the subject during the continuous intravenous infusion.

The total duration of the treatment course with the neurosteroid (e.g., ganaxolone) typically will not exceed 10 hours. Although, those skilled in the art will appreciate that in some instances, the subject will benefit from a treatment course with the neurosteroid (e.g., ganaxolone) that exceeds 10 hours.

The treatment course can achieve a ganavolone plasma concentration of about 400 ng/ml to about 1000 ng/ml.

Additional description of the method and guidance for the practice of the method are provided herein. For ease of presentation, further details and guidance are provided with respect to a preferred aspect using ganaxolone. It is intended that the further details and guidance also relate to treatment with other neurosteroids.

### A. Intravenous Bolus

As described above, the methods disclosed herein comprise administering to a subject in need thereof a therapeutically effective course of a neurosteroid (e.g., ganaxolone) as an intravenous bolus plus continuous intravenous infusion.

The intravenous bolus of neurosteroid (i.e., ganaxolone) is administered in an amount sufficient to suppress SE, which typically is an amount sufficient to produce a ganaxolone plasma concentration of at least about 400 ng/ml to about 1000 ng/ml. Preferably, the intravenous bolus of ganaxolone results in minimal or no anesthetic effects. For example, preferably, the amount of neurosteroid (e.g., ganaxolone) administered does not result in loss of consciousness, does not result in muscle paralysis, and/or does not cause deep sedation. Preferably, treatment in accordance with the methods described herein does not require the subject to undergo controlled ventilation and/or endotracheal intubation.

The intravenous bolus of neurosteroid (e.g., ganavolone) during each course of medication can be administered (i.e., infused) into the subject at an amount of about 1 mg, about 2 mg, about 3 mg, about 4 mg/hr, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. Preferably, about 20 mg of ganaxolone is infused into the subject during the intravenous bolus. Although, in some instances, the prescribing clinician will recognize that the subject may benefit from a lower or higher dose of ganaxolone. In some instances, about 15 mg of ganaxolone is infused into the subject during the intravenous bolus. In some instances, about 30 mg of ganaxolone is infused into the subject during the intravenous bolus.

In subjects weighing less than about 40 kg, about 0.10 mg/kg, about 0.11 mg/kg, about 0.12 mg/kg, about 0.13 mg/kg, about 0.14 mg/kg, about 0.15 mg/kg, about 0.16 mg/kg, about 0.17 mg/kg, about 0.18 mg/kg, about 0.19 mg/kg, about 0.20 mg/kg, about 0.21 mg/kg, about 0.22 mg/kg, about 0.23 mg/kg, about 0.24 mg/kg, about 0.25 mg/kg, about 0.26 mg/kg, about 0.27 mg/kg, about 0.28 mg/kg, about 0.29 mg/kg, about 0.30 mg/kg, about 0.31 mg/kg, about 0.32 mg/kg, about 0.33 mg/kg, about 0.34 mg/kg, about 0.35 mg/kg, about 0.36 mg/kg, about 0.37 mg/kg, about 0.38 mg/kg, about 0.39 mg/kg, about 0.40 mg/kg, about 0.41 mg/kg, about 0.42 mg/kg, about 0.43 mg/kg, about 0.44 mg/kg, about 0.45 mg/kg, about 0.46 mg/kg, about 0.47 mg/kg, about 0.48 mg/kg, about 0.49 mg/kg, about 0.5 mg/kg, about 0.51 mg/kg, about 0.52 mg/kg, about 0.53 mg/kg, about 0.54 mg/kg, about 0.55 mg/kg, about 0.56 mg/kg, about 0.57 mg/kg, about 0.58 mg/kg, about 0.59 mg/kg, about 0.60 mg/kg, about 0.61 mg/kg, about 0.62 mg/kg, about 0.63 mg/kg, about 0.64 mg/kg, about 0.65 mg/kg, about 0.66 mg/kg, about 0.67 mg/kg, about 0.68 mg/kg, about 0.69 mg/kg, about 0.7 mg/kg, about 0.75 mg/kg, about 0.80 mg/kg, about 0.85 mg/kg, about 0.90 mg/kg, about 0.95 mg/kg of neurosteroid (e.g., ganaxolone) can be infused into the subject during the intravenous bolus for each course of medication.

The intravenous bolus for each course of medication can be administered to the subject for any desired period of time and is typically administered from about 1 minute to about 10) minutes, such as, from about 1 minute to about 5 minutes, about 1 minute to about 4 minutes, about 1 minute to about 3 minutes, about 1 minute to about 2 minutes, about 2 minutes to about 5 minutes, about 2 minutes to about 4 minutes, about 2 minutes to about 3 minutes, about 3 minutes to about 5 minutes, or about 3 minutes to about 4 minutes. The intravenous bolus can preferably be administered to the subject for about 1 minute, about 2 minutes, about 3 minutes, about 4 minutes, or about 5 minutes. More preferably, the intravenous bolus is administered to the subject for about 3 minutes.

The intravenous bolus administered during the one or more additional courses can be the same as the first course. The intravenous bolus administered during the one or more additional courses can be different to the first course.

In some instances, the one or more additional courses can optionally comprise administering an intravenous bolus to a subject. In some instances, an intravenous bolus will not be administered to a subject that shows signs of seizure re-lapse within 30 minutes from completion of a course of neurosteroid (e.g., ganaxolone).

### B. Continuous Intravenous Infusion

The continuous intravenous infusion of ganaxolone is administered periprocedural with the intravenous bolus. For instance, the continuous intravenous infusion can be initiated concurrently with the administration of the intravenous bolus. Alternatively, the continuous intravenous infusion can be initiated before or after the administration of the intravenous bolus. Typically, the intravenous infusion and continuous intravenous infusion is administered from the same ganaxolone source (e.g., and intravenous bag connect to IV line) and are initiated concurrently. The continuous intravenous infusion of neurosteroid (e.g., ganaxolone) is administered in an amount to continue SE suppression throughout the treatment period and beyond. The continuous intravenous infusion of ganaxolone provides durable suppression of SE that lasts preferably through the treatment period and preferably post-treatment.

In embodiments, the continuous intravenous infusion of ganaxolone achieves suppression of SE for at least 1 hour, at least 2 hours, at least 3 hours, at least 4 hours, at least 5 hours, at least 6 hours at least 7 hours, at least 8 hours, at least 9 hours, at least 10 hours, at least 11 hours, at least 12 hours, at least 1 day, at least 2 days, at least 3 days, at least 4 days, at least 5 days, at least 6 days, at least 7 days or longer post-treatment. In embodiments, the continuous intravenous infusion of ganaxolone achieves suppression of SE for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks or longer post-treatment. In embodiments, the continuous intravenous infusion of ganaxolone achieves suppression of SE for at least 1 month, at least 2 months, at least 3 months, at least 4 months, at least 5 months, at least 6 months, at least 7 months, at least 8 months, at least 9 months, at least 10 months, at least 11 months, at least 12 months or longer post-treatment.

The continuous intravenous infusion for each medication course comprises infusing into the subject about 40 mg to about 80 mg of ganaxolone per hour. For example, about 50) mg, about 51 mg, about 52 mg, about 53 mg, about 54 mg, about 55 mg, about 56 mg, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, or about 70) mg, of ganaxolone per hour during the continuous intravenous infusion can be infused. Preferably, the continuous intravenous infusion comprises infusing into the subject about 60 mg of ganaxolone per hour is infused into the subject.

The continuous intravenous infusion can be administered (i.e., infused) into the subject for about 2 hours to about 10 hours (e.g., about 2 hours, about 3 hours, about 4 hours, about 5 hours, about 6 hours, about 7 hours, about 8 hours, about 9 hours, about 10 hours) after which the continuous intravenous infusion is terminated.

The continuous intravenous infusion of ganaxolone can be administered to the subject in an amount to achieve a ganavolone plasma concentration of about 400 ng/ml to about 1000 ng/ml. A plasma concentration of ganaxolone above about 1000 ng/ml can induce anesthesia in a subject, which is generally not an intended effect or desired outcome of the methods described herein. Preferably, the continuous intravenous infusion of ganaxolone results in minimal or no anesthetic effects. For example, preferably, the amount of ganaxolone administered does not result in loss of consciousness, does not result in paralysis, and/or does not cause deep sedation compromising respiratory status. Preferably, treatment in accordance with the methods described herein does not require the subject to undergo controlled ventilation and/or endotracheal intubation.

During continuous intravenous infusion of ganaxolone, one or more additional intravenous bolus of ganaxolone can be administered to a subject that shows signs of SE re-lapse or experiences SE re-lapse. Electroencephalogram (EEG) can be used to detect signs of SE re-lapse. Plasma concentration of ganaxolone can alternatively or in combination with EEG be used to detect signs of SE re-lapse or a subject that experiences SE re-lapse. For example, a ganaxolone plasma concentration below 400 ng/ml can suggest that the subject is likely to re-lapse.

### C. Methods for Treating

The disclosure relates to methods for treating ESE. The methods disclosed herein comprise administering to a subject in need thereof a therapeutically effective course of a neurosteroid, preferably ganaxolone, as an intravenous bolus and a continuous intravenous infusion. The intravenous bolus is administered in an amount to suppress SE. Suppression of SE reduces seizure burden. The continuous intravenous infusion is administered in an amount for continued SE suppression.

Treatment with the course of neurosteroid (e.g., ganaxolone) typically last about 2 hours to about 10 hours, (e.g., about 2 hours, about 3 hours, about 4 hours, about 5 hours, about 6 hours, about 7 hours, about 8 hours, about 9 hours, about 10 hours) after which the continuous intravenous infusion is terminated. In preferred aspects, the course of neurosteroid (e.g., ganaxolone) lasts about 2 hours to about 4 hours. In preferred aspects the course of neurosteroid (e.g., ganaxolone) lasts about 2 hours. In preferred aspects, the course of neurosteroid (e.g., ganaxolone) lasts about 4 hours.

The methods disclosed herein can further comprise administering to a subject in need thereof one or more additional courses of medication with ganaxolone administered as an intravenous bolus plus continuous intravenous infusions. Generally, if the subject shows signs of seizure re-lapses within about 12 hours from the initiation of a course of neurosteroid, the subject can be administered a second course of neurosteroid, typically for treatment period that is longer than the first course. For example if the first course treatment period was about 2 hours, the second course treatment period can be for about 4 hours to about 8 hours. Preferably, the second course treatment period is about 4 hours.

A third course of neurosteroid (e.g., ganaxolone), can be administered if the subject shows signs of seizure re-lapse within about 12 hours from the initiation from the second course of neurosteroid. Typically, the third course of neurosteroid is administered for a similar treatment period as the second course. For example, if the first course treatment period was about 2 hours and the second course treatment period was about 4 hours, the third treatment period can be for about 4 hours. Preferably, the third treatment period is about 4 hours.

The treatment course disclosed herein typically does not exceed 10 hours. With all courses of neurosteroid (e.g., ganaxolone) preferably does not exceed 10 hours. For example, the first course can be about 2 hours, the second course can be about 4 hours, and the third course can be about 4 hours. As another example, the first course can be about 2 hours and the second course can be about 8 hours.

SE presents as a prolong seizure for a period of at least 5 minutes or without recovery between seizures. Suppression of SE typically breaks the seizures (i.e., suppresses or reduces the seizure activity). Clinically, suppression of SE can be reduction in seizure burden (i.e., the percent of time during which there is electrographic seizure activity). For instance, a clinician may consider a seizure burden less than 20% suppression of SE and/or a seizure burden that is at least 50% less than during the 30 minutes prior to the initiation of treatment (i.e., intravenous bolus plus continuous intravenous infusion).

The human subject might be male, female, adults, and children, seniors (65 and older). The human subject may be, e.g., from about 1 year to about 120 years old, from about 1 year to about 100 years old, from about 2 years to about 95 years old, from about 5 years to about 90 years old, from about 7 years to about 85 years old, from about 10 years old to about 85 years old, from about 12 years old to about 85 years old, from about 14 years old to about 85 years old, from about 16 years old to about 85 years old, from about 18 years old to about 85 years old, or from about 20 years old to about 85 years old.

A subject suitable for treatment according to the methods described herein has and/or is experiencing ESE. SE is particularly suitable for the treatment according to the methods described herein.

Treatment of SE typically occurs in stages, first-line, second-line, and third-line. The first-line standard of case treatment is parental benzodiazepines. Exemplary benzodiazepines include but are not limited to clonazepam, lorazepam, midazolam, and diazepam. Benzodiazepines are ineffective in about 35%-45% of cases. If SE continues despite treatment with benzodiazepines, other anti-seizure medications (e.g., fosphenytoin, levetiracetam, and valproate) are administered as a second-line treatment. Second-line treatment is ineffective in over 50% of subjects with ESE.

In embodiments, the ESE subject to be treated in accordance with the methods provided herein has failed first-line treatment. In other embodiments, the SE subject to be treated has failed first-line treatment. In other embodiments, the ESE subject to be treated has failed first-line treatment and second-line treatment. A subject having ESE can be treated according to the methods provided herein prior to receiving other treatments, such as standard of care first-line or second-line treatments. Alternatively the subject can be treated according to the methods provided herein after failure of first-line treatment (e.g., benzodiazepines). In some instances, the subject is to be treated according to the methods provided herein after failure of second-line treatment (e.g., an anti-seizure drug). The subject to be treated according to the methods provided herein may have failed one or more anti-seizure drugs. The subject to be treated according to the methods disclosed herein may have failed first-line treatment (e.g., benzodiazepine) and two or more second line second-line treatments. For instance, the subject may have failed two or more anti-seizure drugs. Exemplary anti-seizure drugs can include, but are not limited to, fosphenytoin/phenytoin, valproic acid, levetiracetam, lacosamide, or brivaracetam.

In certain embodiments, the subject does not have a refractory genetic condition selected from the group consisting of PCDH19-related epilepsy, CDKL5 Deficiency Disorder (CDD), Dravet Syndrome, Lennox-Gastaut syndrome (LGS), Continuous Sleep Wave in Sleep (CSWS), Epileptic Status Epilepticus in Sleep (ESES), and other intractable and refractory genetic epilepsy conditions that clinically resemble PCDH19-related epilepsy, CDKL5 Deficiency Disorder, Dravet Syndrome, LGS, CSWS, and ESES. In certain embodiments, the subject does not have CDKL5 gene disorder. In certain embodiments, the subject does not have PCDH19-related epilepsy.

### D. Ganaxolone

Ganaxolone (alternatively known as 3α-hydroxy-3β-methyl-5α-pregnan-20-one, SPT3162, MD 9150000, CCD-1042, Mepalon, and 1042) is the subject of Investigational New Drug Application (IND) No. 129,433. The molecular formula of ganaxolone is C₂₂H₃₆O₂, and the chemical structure is:

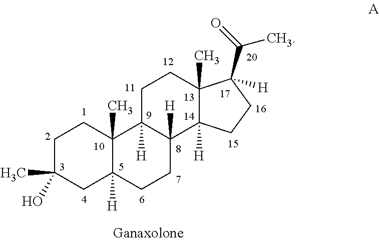

Ganaxolone (3α-hydroxy-3β-methyl-5α-pregnan-20-one) is a 3β-methylated synthetic analogue of the endogenous neurosteroid allopregnanolone with similar biological activity (Carter et al., (1997), The Journal of Pharmacology and Experimental Therapeutics, 280:1284-1295), but it is designed to not activate nuclear (classical) progesterone receptors. Also, in contrast to allopregnanolone, ganaxolone is orally bioavailable.

Ganaxolone acts as a positive allosteric modulator of γ-aminobutyric acid type A (GABAA) receptors in the CNS (Carter et al 1997). Ganaxolone affects GABAA receptors by interacting with a recognition site that is distinct from other allosteric GABAA receptor modulators, such as benzodiazepines and barbiturates. Ganaxolone binds to synaptic- and extrasynaptic receptors, mediating both phasic and tonic modulation, respectively. The unique binding of ganaxolone to these two distinct receptor types does not lead to the tolerance seen with benzodiazepines (Mares and Stehlikova (2010) Neurosci. Let. 469:396-399) and allows ganaxolone to act as a broad-spectrum GABAergic compound with the potential to treat the myriad of symptoms related to pediatric genetic epilepsies, refractory seizures, cognitive and behavioral disorders, and sleep dysfunction.

Ganaxolone provides an alternative mechanism in the treatment of seizures and could serve as effective therapy in the management of SE, including generalized convulsive status epilepticus, non-convulsive status epilepticus, early status epilepticus, established status epilepticus, refractory status epilepticus, or super-refractory status epilepticus.

Ganaxolone does not activate the progesterone receptor directly or indirectly, via metabolic conversion, confirming ganaxolone's lack of hormonal activity.

Ganaxolone has the advantage of controlling both convulsive and non-convulsive seizures. In experimental animals, Ganaxolone produced immediate and prolonged cessation of benzodiazepine-resistant SE as evidenced by a block of convulsions, reduction of EEG seizure activity, and increased survival.

Ganaxolone is insoluble in water. Its solubility in 95% alcohol, propylene glycol and polyethylene glycol are 13 mg/mL. 3.5 mg/mL, and 3.1 mg/mL, respectively. Ganaxolone has a relatively long half-life—approximately 20 hours in human plasma following oral administration (Nohria, V. and Giller, E., Neurotherapeutics, (2007) 4(1): 102-105). Furthermore, ganaxolone has a short Tₘₐₓ, which means that therapeutic blood levels are reached quickly.

Ganaxolone is metabolized by CYP3A4/5, and in vitro data and human PK data from subjects taking strong CYP inducers (carbamazepine and phenytoin) has shown increased ganaxolone clearance with approximately a 45% lowering in overall ganavolone levels and exposure.

In the ganaxolone development program overall, no clinically significant trends in electrocardiogram (ECG) intervals, vital signs, or physical or neurological examinations have been noted, and no mean changes from baseline in clinical laboratory results have been identified. In the completed placebo-controlled Phase 1, 2, and 3 studies, 0.32% of subjects who received ganaxolone and 0.46% of subjects who received placebo developed elevated LFTs during the study (>3×ULN AST and/or ALT). A subject participating in the ganaxolone paediatric epilepsy study developed liver failure, which was not considered to be related to ganaxolone. The subject was diagnosed with short bowel syndrome, liver steatosis and IgG-cholangitis, which were considered to be the causal factors for the subject's liver failure. There have been no other cases of Hy's Law or liver failure in the ganaxolone development program. It is known that ganaxolone and its metabolites are excreted to breast milk. After cessation of the dosing, plasma ganaxolone levels are expected to drop rapidly, but it is possible that low sub-therapeutic levels persist for several days as ganaxolone is slowly released from tissues.

Previous toxicology studies in animals focusing on prenatal and neonatal development have not demonstrated toxicities associated with ganaxolone. Ganaxolone has been administered to infants with severe forms of epilepsy as early as 4 months of age. In clinical trials involving administration of ganaxolone over several weeks, the study drug has been tapered off over a 1 to 2-week period. There have been no reports of withdrawal symptoms emerging after cessation of ganaxolone.

### E. Formulations

Contemplated herein are formulations that comprise a therapeutically effective amount of a neurosteroid for treating status epilepticus according the methods disclosed herein. Preferably the neurosteroid is ganaxolone. Other neurosteroid that can be used according to the methods disclosed herein include, but are not limited to allopregnanolone, 3α-Dihydroprogesterone, 5α-Dihydroprogesterone, 5β-Dihydroprogesterone, Allopregnanediol, Dihydrodeoxy corticosterone, Pregnanediol, Pregnanolone, Tetrahydrodeoxy corticosterone, Alfadolone, Alfadolone acetate, EIDD-036, Hydroxydione, Minaxolone, 21-chloro-2β-morpholin-4-yl-5β-pregnan-3α-ol-20-one, 2β-(2,2-dimethyl-4-morpholinyl)-3α-hydroxy-11,20-dioxo-5α-pregnan-21-yl methanesulfonate, or Renanolone, SGE-516, SGE-872, SAGE-217 (Zuranolone: 3α-hydroxy-3β-methyl-21-(4-cyano-1H-pyrazol-1′-yl)-19-nor-5β-pregnan-20-one).

The formulation is preferably an intravenous formulation of ganaxolone. The intravenous formulation of ganaxolone can comprise a cyclodextrin (e.g., a sulfobutyl ether β-cyclodextrin (Captisol®). The IV solution can comprise a sterile ready to administer solution containing 1 mg/ml ganaxolone in Captisol® (Captisol®:GNX ratio 60:1). The ready to administer solution can comprise 1 mg/ml ganaxolone in sulfobutyl ether β-cyclodextrin (Captisol®) having a Captisol to ganaxolone ratio of 60:1, and a buffer (i.e., phosphate and/or sodium chloride). In embodiments, the IV solution is a sterile solution containing 3 mg/ml ganaxolone in Captisol® (Sulfobutylether-β-Cyclodextrin) (Captisol®:GNX ratio 70:1) or 5 mg/ml ganaxolone in Captisol, each of which may or may not be may be diluted with 0.9% saline (i.e., sodium chloride) solution, for example to produce a 1 mg/ml ganaxolone solution for administration, prior to administration.

In certain embodiments, the formulation (e.g., an intravenous formulation) comprises ganaxolone and sulfobutylether-β-cyclodextrin (e.g., Captisol®) in a weight ratio from about 1:50 to about 1:75. In some of these embodiments, the weight ratio ganaxolone and Captisol® is about 1:51, about 1:52, about 1:53, about 54:1, about 1:55, about 1:56, about 1:57, about 1:58, about 1:59, about 1:60, about 1:61, about 1:62, about 1:63, about 1:64, about 1:65, about 1:66, about 1:67, about 1:68, about 1:69, about 1:70, about 1:71, or about 1:72. In some of these embodiments, the weight ratio ganaxolone and Captisol® is about 1:60.

The intravenous formulation may be selected, e.g., from the group consisting of nanocrystal formulations; emulsions; lyocells; solvents or surfactants; liposomes; microemulsions; and liquids containing solid-lipid nanoparticles.

In certain embodiments, the intravenous formulation is an IV solution. An intravenous formulation is preferably a sterile liquid (e.g., aqueous liquid in the form of an emulsion, a suspension, a solution and the likes). In some of these embodiments, the IV solution comprises ganaxolone and a pharmaceutically acceptable solvent(s) and/or oil(s) that can solubilize ganaxolone.

In certain embodiments, the intravenous formulation is an oil-in-water emulsion.

In certain embodiments, the intravenous formulation is a liquid nanoparticulate formulation (e.g., a liquid comprising nanoparticles of ganaxolone). In some of the embodiments, the nanoparticulate formulation comprises ganaxolone and a polymeric and/or ionic stabilizer, and is free from complexing agents. In certain embodiments, the polymeric and ionic stabilizers are selected from the group consisting of surfactants. In certain embodiments, surfactants are selected from the group consisting of sorbitan esters, polyoxyethylene sorbitan fatty acid esters, poloxamers, cholesterol salts, and bile salts.

In certain embodiments, the formulation for the intravenous infusion may be a formulation as described and prepared in U.S. Patent Publication No. 2017/0258812 or U.S. Patent Publication No. 2016/0228454. However, formulations for the intravenous infusion may be prepared in accordance with other methods known to those skilled in the art.

As described in U.S. Patent Publication No. 2016/0228454, an aqueous injectable ganaxolone formulation may comprise a) ganaxolone and sulfobutyl ether-β-cyclodextrin in an inclusion complex; and b) water. In some embodiments, the complex comprising ganaxolone and sulfobutyl ether-β-cyclodextrin comprises a 1:1 ganaxolone:sulfobutyl ether-O-cyclodextrin complex; and the w/w ratio of sulfobutyl ether-β-cyclodextrin to ganaxolone is about 52:1 or greater. In some embodiments, the formulation may further comprise surfactant. In some embodiments, the surfactant is a sorbitan ester, a polyoxyethylene sorbitan fatty acid ester, a poloxamer, a cholesterol salt, or a bile salt. In some embodiments, the surfactant may comprise from about 1 to about 15 percent of the formulation by weight.

In some embodiments, the surfactant is polysorbate 80. In some embodiments, the formulation further comprises a buffer and has a pH of about 6.0 to about 7.6. In some embodiments, the buffer is a phosphate buffer. In some embodiments, the buffer is a combination of a monobasic phosphate buffer and a dibasic phosphate buffer, wherein the concentration of each phosphate buffer is 2 mM to 50 mM. In some embodiments, the buffer is a phosphate buffer. In some embodiments, the buffer is a combination of a monobasic phosphate buffer and a dibasic phosphate buffer, wherein the concentration of each phosphate buffer is 2 mM to 50 mM. In some embodiments, the concentration of ganaxolone is 2 mg/ml to 8 mg/ml, the w/w ratio of sulfobutyl ether-β-cyclodextrin to ganaxolone is within the range from about 52:1 to about 90:1; the formulation contains a buffer and has a pH of 6.7 to 7.3 or a pH of 6.0 to 7.0; and the formulation contains from 1 to 15 weight percent surfactant. In some embodiments, the concentration of ganaxolone is 1 mg/ml to 5 mg/ml; the weight percent of sulfobutyl ether-β-cyclodextrin 25% to 35%; and the formulation contains from 5% to 15% (weight percent) of at least one of the following: a surfactant, ethanol, glycerin, or propylene glycol. In some embodiments, the formulation further comprises a preservative. In some embodiments, the preservative is benzyl alcohol, chlorbutanol, 2-ethoxyethanol, parabens (including methyl, ethyl, propyl, butyl, and combinations), benzoic acid, sorbic acid, chlorhexidene, phenol, 3-cresol, thimerosal, or a phenylmercurate salt.

As further described in U.S. Patent Publication No. 2016/0228454, the formulation may be a lyophilized ganaxolone formulation comprising ganaxolone and sulfobutyl ether-β-cyclodextrin, wherein the ganaxolone formulation is 1.0% to 1.5% ganaxolone. In some embodiments, the formulation may further comprise a bulking agent. In some embodiments, the bulking agent is mannitol, lactose, sucrose, trehalose, sorbitol, glucose, rafinose, glycine, histidine, polyethylene glycol (PEG), or polyvinyl pyrrolidone (PVP).

Ganaxolone formulations suitable for parenteral administration in the methods of the present invention may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, ganaxolone can be dissolved at concentrations of >1 mg/ml using water soluble beta cyclodextrins (e.g. beta-sulfobutyl-cyclodextrin and 2-hydroxypropylbetacyclodextrin). A particularly suitable cyclodextrin is a substituted-β-cyclodextrin is Captisol®. Proper fluidity can be maintained, for example, by the use of a coating such as lecithin, by the maintenance of the required particle size in the case of dispersions, and by the use of surfactants. Ganaxolone formulations suitable for subcutaneous injection may also contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, sorbic acid, and the like. It may also be desirable to include isotonic agents, such as sugars, sodium chloride, and the like. Prolonged drug absorption of the injectable pharmaceutical form can be brought about by the use of agents delaying absorption, such as aluminum monostearate and gelatin. Ganaxolone suspension formulations designed for extended release via subcutaneous or intramuscular injection can avoid first pass metabolism and lower dosages of ganaxolone will be necessary to maintain plasma levels of about 400 ng/ml or higher. In such formulations, the particle size of the ganaxolone particles and the range of the particle sizes of the ganaxolone particles can be used to control the release of the drug by controlling the rate of dissolution in fat or muscle.

In certain embodiments, the intravenous formulation is a solution comprising a complexing agent(s). In some of these embodiments, a complexing agent is a molecule with a lipophilic core and hydrophilic outer shell capable of solubilizing ganaxolone.

In certain embodiments, the formulation is an IV solution comprising ganaxolone and sulfobutylether cyclodextrin (Captisol®), wherein ganaxolone is solubilized in sulfobutylether cyclodextrin (Captisol®). In some embodiments, the solution comprises 3 mg of ganaxolone per 1 ml of the solution and is sterile. In certain embodiments, the solution is stable for at least 18 months, is stored refrigerated at a temperature from about 4° C. to about 8° C.

In certain embodiments, the liquid formulation of the present invention may be a formulation as described and prepared in U.S. Pat. No. 8,022,054, entitled “Liquid Ganaxolone Formulations and Methods for the Making and Use Thereof”, hereby incorporated by reference in its entirety. However, the oral liquid (e.g., suspension) formulation of ganaxolone may be prepared in accordance with other methods known to those skilled in the art.

As described in U.S. Pat. No. 8,022,054, the liquid formulation may be an aqueous dispersion of stabilized particles comprising ganaxolone, a hydrophilic polymer, a wetting agent, and an effective amount of a complexing agent that stabilizes particle growth after an initial particle growth and endpoint is reached, the complexing agent selected from the group of small organic molecules having a molecular weight less than 550 and containing a moiety selected from the group consisting of a phenol moiety, an aromatic ester moiety and an aromatic acid moiety, wherein the stabilized particles have a volume weighted median diameter (D50) of the particles from about 50 nm to about 500 nm, the complexing agent being present in an amount from about 0.05% to about 5%, w/w based on the weight of particles, the particles dispersed in an aqueous solution which further contains at least two preservatives in an amount sufficient to inhibit microbial growth. The hydrophilic polymer may be in an amount from about 3% to about 50%, w/w, based on the weight of the solid particles. The wetting agent may be an amount from about 0.01% to about 10%, w/w, based on the weight of the solid particles. Ganaxolone may be in an amount from about 10% to about 80% (and in certain embodiments form about 50% to about 80%) based on the weight of the stabilized particles. The stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. The stabilized particles may exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm.

The complexing agent can be any molecule with a lipophilic core and hydrophilic outer shell capable of solubilizing ganaxolone. In certain embodiments, complexing agent can be a substance containing a phenol moiety, an aromatic ester moiety or an aromatic acid moiety. In certain embodiments, complexing agents are selected from the group consisting of parabens, organic acids, carboxylic acids, aromatic acids, aromatic esters, acid salts of amino acids, methyl anthranilate, sodium metabisulphite, ascorbic acid and its derivatives, malic acid, isoascorbic acid, citric acid, tartaric acid, sodium sulphite, sodium bisulphate, tocopherol, water- and fat-soluble derivatives of tocopherol, sulphites, bisulphites and hydrogen sulphites, para-aminobenzoic acid and esters, 2,6-di-t-butyl-alpha-dimethylamino-p-cresol, t-butylhydroquinone, di-t-amylhydroquinone, di-t-butylhydroquinone, butylhydroxytoluene (BHT), butylhydroxyanisole (BHA), pyrocatechol, pyrogallol, propyl/gallate, nordihydroguaiaretic acid, phosphoric acids, sorbic and benzoic acids, esters, ascorbyl palmitate, derivatives and isomeric compounds thereof, pharmaceutically acceptable salts thereof, and mixtures thereof. In certain embodiments, the complexing agent is selected from the group consisting of a paraben, benzoic acid, phenol, sodium benzoate, methyl anthranilate, and the like. The hydrophilic polymer may be a cellulosic polymer, a vinyl polymer and mixtures thereof. The cellulosic polymer may be a cellulose ether, e.g., hydroxypropymethylcellulose. The vinyl polymer may be polyvinyl alcohol, e.g., vinyl pyrrolidone/vinyl acetate copolymer (S630). The wetting agent may be sodium lauryl sulfate, a pharmaceutically acceptable salt of docusate, and mixtures thereof. The aqueous dispersion may further comprise a sweetener, e.g., sucralose. In certain embodiments, the preservative is selected from the group consisting of potassium sorbate, methylparaben, propylparaben, benzoic acid, butylparaben, ethyl alcohol, benzyl alcohol, phenol, benzalkonium chloride, and mixtures of any of the foregoing.

In some embodiments, liquid ganaxolone formulations are provided comprising the ganaxolone particles described herein and at least one dispersing agent or suspending agent for oral administration to a subject. The ganaxolone formulation may be a powder and/or granules for suspension, and upon admixture with water, a substantially uniform suspension is obtained. As described herein, the aqueous dispersion can comprise amorphous and non-amorphous ganaxolone particles of consisting of multiple effective particle sizes such that ganaxolone particles having a smaller effective particle size are absorbed more quickly and ganaxolone particles having a larger effective particle size are absorbed more slowly. In certain embodiments, the aqueous dispersion or suspension is an immediate release formulation. In another embodiment, an aqueous dispersion comprising amorphous ganaxolone particles is formulated such that about 50% of the ganaxolone particles are absorbed within about 3 hours after administration and about 90% of the ganaxolone particles are absorbed within about 10 hours after administration. In other embodiments, addition of a complexing agent to the aqueous dispersion results in a larger span of ganaxolone containing particles to extend the drug absorption phase such that 50-80% of the particles are absorbed in the first 3 hours and about 90% are absorbed by about 10 hours.

A suspension is “substantially uniform” when it is mostly homogenous, that is, when the suspension is composed of approximately the same concentration of ganaxolone at any point throughout the suspension. Preferred embodiments are those that provide concentrations essentially the same (within 15%) when measured at various points in a ganaxolone aqueous oral formulation after shaking. Especially preferred are aqueous suspensions and dispersions, which maintain homogeneity (up to 15% variation) when measured 2 hours after shaking. The homogeneity should be determined by a sampling method consistent with regard to determining homogeneity of the entire composition. In one embodiment, an aqueous suspension can be re-suspended into a homogenous suspension by physical agitation lasting less than 1 minute. In another embodiment, an aqueous suspension can be re-suspended into a homogenous suspension by physical agitation lasting less than 45 seconds. In yet another embodiment, an aqueous suspension can be re-suspended into a homogenous suspension by physical agitation lasting less than 30 seconds. In still another embodiment, no agitation is necessary to maintain a homogeneous aqueous dispersion.

In some embodiments, ganaxolone formulations are powders for aqueous dispersion and comprise stable ganaxolone particles having an effective particle size by weight of less than 500 nm formulated with ganaxolone particles having an effective particle size by weight of greater than 500 nm. In such embodiments, the formulations have a particle size distribution wherein about 10% to about 100% of the ganaxolone particles by weight are between about 75 nm and about 500 nm, about 0% to about 90% of the ganaxolone particles by weight are between about 150 nm and about 400 nm, and about 0% to about 30% of the ganaxolone particles by weight are greater than about 600 nm. The ganaxolone particles describe herein can be amorphous, semi-amorphous, crystalline, semi-crystalline, or mixture thereof.

In one embodiment, the aqueous suspensions or dispersions described herein comprise ganaxolone particles or ganaxolone complex at a concentration of about 20 mg/ml to about 150 mg/ml of suspension. In another embodiment, the aqueous oral dispersions described herein comprise ganaxolone particles or ganaxolone complex particles at a concentration of about 25 mg/ml to about 75 mg/ml of solution. In yet another embodiment, the aqueous oral dispersions described herein comprise ganaxolone particles or ganaxolone complex at a concentration of about 50 mg/ml of suspension. The aqueous dispersions described herein are especially beneficial for the administration of ganaxolone to infants (less than 2 years old), children under 10 years of age and any patient group that is unable to swallow or ingest solid oral dosage forms.

Liquid ganaxolone formulation for oral administration can be aqueous suspensions selected from the group including, but not limited to, pharmaceutically acceptable aqueous oral dispersions, emulsions, solutions, and syrups. See, e.g., Singh et al., Encyclopedia of Pharmaceutical Technology, 2nd Ed., pp. 754-757 (2002). In addition to ganaxolone particles, the liquid dosage forms may comprise additives, such as: (a) disintegrating agents; (b) dispersing agents; (c) wetting agents; (d) at least one preservative, (e) viscosity enhancing agents, (f) at least one sweetening agent, (g) at least one flavoring agent, (h) a complexing agent, and (i) an ionic dispersion modulator. In some embodiments, the aqueous dispersions can further comprise a crystalline inhibitor.

Examples of disintegrating agents for use in the aqueous suspensions and dispersions include, but are not limited to, a starch, e.g., a natural starch such as corn starch or potato starch, a pregelatinized starch such as National 1551 or AMIJELE®, or sodium starch glycolate such as PROMOGEL® or EXPLOTAB®; a cellulose such as a wood product, microcrystalline cellulose, e.g., AVICEL®, AVICEL® PH101, AVICEL® PH102, AVICEL® PH105, ELCEMA® P100, EMCOCEL®, VIVACEL®, MING TIA®, and SOLKA-FLOC®, methylcellulose, croscarmellose, or a cross-linked cellulose, such as cross-linked sodium carboxymethylcellulose (AC-DI-SOL®), cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay such as VEEGUM® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

In some embodiments, the dispersing agents suitable for the aqueous suspensions and dispersions described herein are known in the art and include, for example, hydrophilic polymers, electrolytes, TWEEN® 60 or 80, PEG, polyvinylpyrrolidone (PVP; commercially known as PLASDONE®), and the carbohydrate-based dispersing agents such as, for example, hydroxypropylcellulose and hydroxypropylcellulose ethers (e.g., HPC, HPC-SL, and HPC-L), hydroxypropylmethylcellulose and hydroxypropylmethylcellulose ethers (e.g. HPMC K100, HPMC K4M, HPMC K15M, and HPMC K100M), carboxymethylcellulose sodium, methylcellulose, hydroxyethylcellulose, hydroxypropylmethylcellulose phthalate, hydroxypropylmethylcellulose acetate stearate, noncrystalline cellulose, magnesium aluminum silicate, triethanolamine, polyvinyl alcohol (PVA), polyvinylpyrrolidone/vinyl acetate copolymer (Plasdone®, e.g., S-630), 4-(1,1,3,3-tetramethylbutyl)-phenol polymer with ethylene oxide and formaldehyde (also known as tyloxapol), poloxamers (e.g., Pluronics F68®, F88®, and F108®, which are block copolymers of ethylene oxide and propylene oxide); and poloxamines (e.g., Tetronic 9080, also known as Poloxamine 9080, which is a tetrafunctional block copolymer derived from sequential addition of propylene oxide and ethylene oxide to ethylenediamine (BASF Corporation, Parsippany, N.J.)). In other embodiments, the dispersing agent is selected from a group not comprising one of the following agents: hydrophilic polymers; electrolytes; TWEEN® 60 or 80; PEG; polyvinylpyrrolidone (PVP); hydroxypropylcellulose and hydroxypropyl cellulose ethers (e.g., HPC, HPC-SL, and HPC-L); hydroxypropyl methylcellulose and hydroxypropyl methylcellulose ethers (e.g. HPMC K100, HPMC K4M, HPMC K15M, HPMC K100M, and Pharmacoat® USP 2910 (Shin-Etsu)); carboxymethylcellulose sodium; methylcellulose; hydroxyethylcellulose; hydroxypropylmethyl-cellulose phthalate; hydroxypropylmethyl-cellulose acetate stearate; non-crystalline cellulose; magnesium aluminum silicate; triethanolamine; polyvinyl alcohol (PVA); 4-(1,1,3,3-tetramethylbutyl)-phenol polymer with ethylene oxide and formaldehyde; poloxamers (e.g., PLURONICS F68®, F88®, and F108®, which are block copolymers of ethylene oxide and propylene oxide); or poloxamines (e.g., Tetronic 908®, also known as Poloxamine 908%).

Wetting agents (including surfactants) suitable for the aqueous suspensions and dispersions described herein are known in the art and include, but are not limited to, acetyl alcohol, glycerol monostearate, polyoxyethylene sorbitan fatty acid esters (e.g., the commercially available Tweens® such as e.g., Tween 20® and Tween 80® (ICI Specialty Chemicals)), and polyethylene glycols (e.g., Carbowaxs 3350® and 1450®, and Carpool 934@ (Union Carbide)), oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, phosphotidylcholine and the like.

Suitable preservatives for the aqueous suspensions or dispersions described herein include, for example, potassium sorbate, parabens (e.g., methylparaben and propylparaben) and their salts, benzoic acid and its salts, other esters of parahydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth. In one embodiment, the aqueous liquid dispersion can comprise methylparaben and propylparaben in a concentration ranging from about 0.01% to about 0.3% methylparaben by weight to the weight of the aqueous dispersion and 0.005% to 0.03% propylparaben by weight to the total aqueous dispersion weight. In yet another embodiment, the aqueous liquid dispersion can comprise methylparaben 0.05 to about 0.1 weight % and propylparaben from 0.01-0.02 weight % of the aqueous dispersion.

Suitable viscosity enhancing agents for the aqueous suspensions or dispersions described herein include, but are not limited to, methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans and combinations thereof. The concentration of the viscosity enhancing agent will depend upon the agent selected and the viscosity desired.

Examples of natural and artificial sweetening agents suitable for the aqueous suspensions or dispersions described herein include, for example, acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet®. Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In one embodiment, the aqueous liquid dispersion can comprise a sweetening agent or flavoring agent in a concentration ranging from about 0.0001% to about 10.0% the weight of the aqueous dispersion. In another embodiment, the aqueous liquid dispersion can comprise a sweetening agent or flavoring agent in a concentration ranging from about 0.0005% to about 5.0% wt % of the aqueous dispersion. In yet another embodiment, the aqueous liquid dispersion can comprise a sweetening agent or flavoring agent in a concentration ranging from about 0.0001% to 0.1 wt %, from about 0.001% to about 0.01 weight %, or from 0.0005% to 0.004% of the aqueous dispersion.

In addition to the additives listed above, the liquid ganaxolone formulations can also comprise inert diluents commonly used in the art, such as water or other solvents, solubilizing agents, and emulsifiers.

In some embodiments, the ganaxolone formulations can be self-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase can be added just prior to administration, which ensures stability of an unstable or hydrophobic active ingredient. Thus, the SEDDS provides an effective delivery system for oral and parenteral delivery of hydrophobic active ingredients. SEDDS may provide improvements in the bioavailability of hydrophobic active ingredients. Methods of producing self-emulsifying dosage forms are known in the art include, but are not limited to, for example, U.S. Pat. Nos. 5,858,401, 6,667,048, and 6,960,563.

Exemplary emulsifiers are ethyl alcohol, isopropyl alcohol, ethyl carbonate, ethyl acetate, benzyl alcohol, benzyl benzoate, propylene glycol, 1,3-butyleneglycol, dimethylformamide, sodium lauryl sulfate, sodium docusate, cholesterol, cholesterol esters, taurocholic acid, phosphotidylcholine, oils, such as cottonseed oil, groundnut oil, corn germ oil, olive oil, castor oil, and sesame oil, glycerol, tetrahydrofurfuryl alcohol, polyethylene glycols, fatty acid esters of sorbitan, or mixtures of these substances, and the like.

In certain preferred embodiments, the liquid pharmaceutical formulation comprising ganaxolone, hydroxypropyl methylcellulose, polyvinyl alcohol, sodium lauryl sulfate, simethicone, methyl paraben, propyl paraben, sodium benzoate, citric acid, and sodium citrate at pH 3.8-4.2. The suspension may comprise ganaxolone at a concentration of 50 mg/ml. The formulation may further comprise a pharmaceutically acceptable sweetener (e.g., sucralose) and/or a pharmaceutically acceptable flavorant (e.g., cherry). The formulation may be enclosed, e.g., in a 120 mL, 180 mL, 240 mL, or 480 mL bottle.

A formulation for oral administration may be an oral solid dosage form (e.g., an oral capsule or tablet) or a liquid (e.g., an oral suspension comprising ganaxolone). In certain embodiments, the oral suspension is administered to the patient via the use of an oral syringe.

In certain embodiments, the liquid formulation of the present invention may be a formulation as described and prepared in Applicant's prior U.S. Pat. No. 8,022,054, entitled “Liquid Ganaxolone Formulations and Methods for the Making and Use Thereof”, hereby incorporated by reference in its entirety. However, the oral liquid (e.g., suspension) formulation of ganaxolone may be prepared in accordance with other methods known to those skilled in the art.

In certain preferred embodiments, the oral solid formulation of the present invention may be a formulation as described and prepared in Applicant's prior U.S. Pat. No. 7,858,609, entitled “Solid Ganaxolone Formulations and Methods for the Making and Use Thereof”, hereby incorporated by reference in its entirety. However, the oral solid dosage formulation of ganaxolone may be prepared in accordance with other methods known to those skilled in the art.

For example, as disclosed in U.S. Pat. No. 7,858,609, the oral solid formulation may comprise stabilized particles comprising ganaxolone, a hydrophilic polymer, a wetting agent, and an effective amount of a complexing agent that stabilizes particle growth after an initial particle growth and endpoint is reached, the complexing agent being a small organic molecule having a molecular weight less than 550 and containing a moiety selected from the group consisting of a phenol moiety, an aromatic ester moiety and an aromatic acid moiety, wherein the stabilized particles have a volume weighted median diameter (D50) of the particles is from about 50 nm to about 500 nm, the complexing agent being present in an amount from about 0.05% to about 5% w/w, based on the weight particles of the solid. The hydrophilic polymer may be in an amount from about 3% to about 50%, w/w, based on the weight of the solid particles. The wetting agent may be an amount from about 0.01% to about 10%, w/w, based on the weight of the solid particles. Ganaxolone may be in an amount from about 10% to about 80% (and in certain embodiments form about 50% to about 80%) based on the weight of the stabilized particles. The stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. The stabilized particles may exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm. The solid stabilized particles may be combined with optional excipients and prepared for administration in the form of a powder, or they may be incorporated into a dosage form selected from the group consisting of a tablet or capsule. The complexing agent may be a paraben, benzoic acid, phenol, sodium benzoate, methyl anthranilate, and the like. The hydrophilic polymer may be a cellulosic polymer, a vinyl polymer and mixtures thereof. The cellulosic polymer may be a cellulose ether, e.g., hydroxypropymethylcellulose. The vinyl polymer may be polyvinyl alcohol, e.g., vinyl pyrrolidone/vinyl acetate copolymer (S630). The wetting agent may be sodium lauryl sulfate, a pharmaceutically acceptable salt of docusate, and mixtures thereof. When the particles are incorporated into a solid dosage form, the solid dosage form may further comprise at least one pharmaceutically acceptable excipient, e.g., an ionic dispersion modulator, a water soluble spacer, a disintegrant, a binder, a surfactant, a plasticizer, a lubricant, a diluent and any combinations or mixtures thereof. The water soluble spacer may be a saccharide or an ammonium salt, e.g., fructose, sucrose, glucose, lactose, mannitol. The surfactant may be, e.g., polysorbate. The plasticizer may be, e.g., polyethylene glycol. The disintegrant may be cross-linked sodium carboxymethylcellulose, crospovidone, mixtures thereof, and the like.

A capsule may be prepared, e.g., by placing the bulk blend ganaxolone formulation, described herein, inside of a capsule. In some embodiments, the ganaxolone formulations (non-aqueous suspensions and solutions) are placed in a soft gelatin capsule. In other embodiments, the ganaxolone formulations are placed in standard gelatin capsules or non-gelatin capsules such as capsules comprising HPMC. In other embodiments, the ganaxolone formulations are placed in a sprinkle capsule, wherein the capsule may be swallowed whole or the capsule may be opened and the contents sprinkled on food prior to eating. In some embodiments of the present invention, the therapeutic dose is split into multiple (e.g., two, three, or four) capsules. In some embodiments, the entire dose of the ganaxolone formulation is delivered in a capsule form.

In certain embodiments, each capsule contains either 200 mg or 225 mg ganaxolone, and hydroxypropyl methylcellulose, sucrose, polyethylene glycol 3350, polyethylene glycol 400, sodium lauryl sulfate, sodium benzoate, citric acid anhydrous, sodium methyl paraben, microcrystalline cellulose, 30% Simethicone Emulsion, gelatin capsules, polysorbate 80, and sodium chloride. In some of the embodiments, the size of the capsule is 00.

Alternatively, the oral dosage forms of the present invention may be in the form of a controlled release dosage form, as described in U.S. Pat. No. 7,858,609.

In certain preferred embodiments, the oral solid formulation of the present invention may be a formulation as described and prepared U.S. Pat. No. 8,367,651.

As described in U.S. Pat. No. 8,367,651, solid stabilized particles may comprise ganaxolone, a hydrophilic polymer, a wetting agent, and an effective amount of a complexing agent that stabilizes particle growth after an initial particle growth and endpoint is reached, the complexing agent being a small organic molecule having a molecular weight less than 550 and containing a moiety selected from the group consisting of a phenol moiety, an aromatic ester moiety and an aromatic acid moiety, wherein the stabilized particles have a volume weighted median diameter (D50) of the particles is from about 50 nm to about 500 nm and the concentration of ganaxolone in the solid stabilized particles is at least 50% by weight. The hydrophilic polymer maybe in an amount from about 3% to about 50%, w/w, based on the weight of the solid particles. The wetting agent may be in an amount from about 0.010% to about 10%, w/w, based on the weight of the solid particles. In some of the embodiments, the stabilized particles exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour as compared to the D50 of the stabilized particles when the particles are dispersed in distilled water under the same conditions, wherein the volume weighted median diameter (D50) of the stabilized particles dispersed in SGF or SIF is less than about 750 nm. In some embodiments, the stabilized particles exhibit an increase in volume weighted median diameter (D50) of not more than about 150% when the formulation is dispersed in 15 mL of SGF or SIF at a concentration of 0.5 to 1 mg ganaxolone/mL as compared to the D50 of the stabilized particles when the particles are dispersed in distilled water under the same conditions, wherein the volume weighted median diameter (D50) of the stabilized particles dispersed in SGF or SIF is less than about 750 nm. In some embodiments, ganaxolone may be present in an amount greater than 50% to about 80%, based on the weight of the particles. In some embodiments, the stabilized particles may exhibit an increase in volume weighted median diameter (D50) of not more than about 150% when the particles are dispersed in simulated gastric fluid (SGF) or simulated intestinal fluid (SIF) at a concentration of 0.5 to 1 mg ganaxolone/mL and placed in a heated bath at 36° to 38° C. for 1 hour, as compared to the D50 of the stabilized particles when the particles are dispersed in distilled water under the same conditions, wherein the volume weighted median diameter (D50) of the stabilized particles dispersed in SGF or SIF is less than about 750 nm. In some embodiments, the solid stabilized particles may be in the form of a powder. In some embodiments, the particles may be incorporated into a dosage form selected from the group consisting of a tablet or capsule. In some embodiments, the volume weighted median diameter (D50) of the stabilized particles dispersed in distilled water is from about 100 nm to about 350 nm. In some embodiments, the complexing agent is selected from the group consisting of parabens, benzoic acid, methyl anthranilate, and pharmaceutically acceptable salts thereof and mixtures thereof. In some embodiments, paraben is selected from the group consisting of methylparaben, ethylparaben, propylparaben, pharmaceutically acceptable salts thereof and mixtures thereof. In some embodiments, the hydrophilic polymer is selected from the group consisting of a cellulosic polymer, a vinyl polymer and mixtures thereof. In some embodiments, the cellulosic polymer is a cellulose ether. In some embodiments, the cellulose ether is hydroxypropylmethylcellulose. In some embodiments, the vinyl polymer is polyvinyl alcohol. In some embodiments, the wetting agent is selected from the group consisting of sodium lauryl sulfate, a pharmaceutically acceptable salt of docusate, and mixtures thereof. Is some embodiments, the particles are incorporated into a solid dosage form, further comprising at least one pharmaceutically acceptable excipient selected from the group consisting of an ionic dispersion modulator, an water soluble spacer, a disintegrant, a binder, a surfactant, a plasticizer, a lubricant, and any combinations or mixtures thereof. In some embodiments, the pharmaceutically acceptable excipient comprises an ionic dispersion modulator. In some embodiments, the ionic dispersion modulator is in an amount from about 1% to about 50%, w/w, based on the weight of the solid particles. In some embodiments, the ionic dispersion modulator is a salt. In some embodiments, the ionic dispersion modulator is an inorganic salt is selected from the group consisting of a magnesium salt, a calcium salt, a lithium salt, a potassium salt, a sodium salt and mixtures thereof. In some embodiments, the ionic dispersion modulator is an organic salt is selected from the group consisting of a citrate salt, a succinate salt, a fumarate salt, a malate salt, maleate salt, a tartrate salt, a glutarate salt, a lactate salt and mixtures thereof. In some embodiments, the pharmaceutically acceptable excipient comprises a water soluble spacer. In some embodiments, the water soluble spacer is in an amount from about 2% to about 60%, w/w, based on the weight of the solid particles. In some embodiments, the water soluble spacer is a saccharide or an ammonium salt. In some embodiments, the saccharide is selected from the group consisting of fructose, sucrose, glucose, lactose, mannitol and mixtures thereof. In some embodiments, the disintegrant is selected from the group consisting of cross-linked sodium carboxymethylcellulose, crospovidone and any combinations or mixtures thereof. In some embodiments, the surfactant is a polysorbate. In some embodiments, the plasticizer is polyethylene glycol. In some embodiments, the solid dosage form is an immediate release dosage form. In some embodiments, the solid dosage form is a controlled release dosage form. In some embodiments, the particles are incorporated into an oral solid dosage form comprising (i) a controlled release component comprising a first portion of the stabilized particles; and a controlled release material, and (ii) an immediate release component comprising a second portion of the stabilized particles, the first and second portion of stabilized particles having a volume weighted median diameter (D50) of from about 50 nm to about 500 nm. In some embodiments, the ratio of ganaxolone in controlled release to immediate release is from about 4:1 to about 1:4. In some embodiments, the dosage form provides a therapeutic effect for about 8 to about 24 hours after administration. In some embodiments, the complexing agent is in an amount from about 0.05% to about 5%, w/w, based on the weight of the solid particles. In some embodiments, the complexing agent comprises methylparaben or a salt thereof. In some embodiments, the complexing agent comprises benzoic acid or a salt thereof. In some embodiments, the complexing agent comprises methyl anthranilate. In some embodiments, the formulation includes from about 200 mg to about 800 mg ganaxolone.

As further described in U.S. Pat. No. 8,367,651, solid stabilized particles may also comprise ganaxolone, a hydrophilic polymer, a wetting agent, and an effective amount of a complexing agent selected from the group of small organic molecules having a molecular weight less than 550 and containing a moiety selected from the group consisting of a phenol moiety, an aromatic ester moiety and an aromatic acid moiety, the stabilized particles having a volume weighted median diameter (D50) of the particles from about 50 nm to about 500 nm, the concentration of ganaxolone in the solid stabilized particles being at least 50% by weight. In some embodiments, ganaxolone is present in an amount greater than 50% to about 80%, based on the weight of the particles. In some embodiments, the particles are incorporated into a dosage form selected from the group consisting of a tablet or capsule. In some embodiments, the complexing agent is selected from the group consisting of parabens, benzoic acid, methyl anthranilate, and pharmaceutically acceptable salts thereof and mixtures thereof.

In certain preferred embodiments, the formulation of the present invention may be a pharmaceutical composition described in U.S. Pat. No. 9,029,355.

In certain embodiments, the composition may comprise the ganaxolone nanoparticles as described above, further in formulations as described in U.S. Pat. No. 9,029,355. In some embodiments, the pharmaceutical composition is a compressed tablet. In some embodiments, the pharmaceutical composition is contained inside a capsule.

### F. Definitions

Various terms relating to aspects of the description are used throughout the specification and claims. Such terms are to be given their ordinary meaning in the art unless otherwise indicated. Other specifically defined terms are to be construed in a manner consistent with the definitions provided herein.

The abbreviation “EEG” means electroencephalography.

The term “modified Salzburg criteria” as used herein refers EEG criteria for nonconvulsive status epilepticus based on the modified Salzburg criteria which defines status epilepticus as continuous epileptiform discharges of greater than 2.5 Hz, or discharges below 2.5 Hz and associated subtle clinical phenomenon. See, Leitinger et al., Lancet Neurol; 15:1054-1062 (2016).

The terms “subject” and “patient” are used interchangeably herein to refer to any animal, such as any mamma, including but not limited to, humans, non-human primates, rodents, and the like. In some embodiments, the mammal is a mouse. In some embodiments, the mammal is a human.

The term “effective amount” or “therapeutically effective amount” as used herein refers to an amount of a compound described herein (e.g., a neurosteroid such as ganaxolone) that is sufficient to effect the intended result, including, but not limited to disease treatment as illustrated below. The “therapeutically effective amount” can be an amount effective to manage ictal activity (e.g., seizure activity), suppress SRSE, allow the patient to recover from a hyperexcitable state, prevents SE-relapse or can provide continued suppression of SRSE.

The therapeutically effective amount can vary depending upon the intended application, or the subject and the disease condition being treated, e.g., the weight and age of the subject, the severity of the disease condition, the manner of administration and the like.

The term “pharmaceutical compositions” as used herein are compositions comprising at least one active agent, such as a compound or salt, solvate, or hydrate of ganaxolone, and at least one other substance, such as a carrier. Pharmaceutical compositions optionally contain one or more additional active agents. When specified, pharmaceutical compositions meet the U.S. FDA's GMP (good manufacturing practice) standards for human or non-human drugs. “Pharmaceutical combinations” are combinations of at least two active agents which may be combined in a single dosage form or provided together in separate dosage forms with instructions that the active agents are to be used together to treat a disorder, such as status epilepticus.

As used herein, the terms “treat,” treatment,” or “treating” and grammatically related terms, refer to an improvement of any sign, symptoms, or consequence of the disease, such as prolonged survival, less morbidity, and/or a lessening of side effects.

### 4. EQUIVALENTS

It will be readily apparent to those skilled in the art that other suitable modifications and adaptions of the methods of the invention described herein are obvious and may be made using suitable equivalents without departing from the scope of the disclosure or the embodiments. Having now described certain compounds and methods in detail, the same will be more clearly understood by reference to the following examples, which are introduced for illustration only and not intended to be limiting.

### 5. EXAMPLES

The present invention is further described by the following examples, which are not intended to be limiting in any way.

### Example 1. A Study to Determine the Dosing Regimen and Assess Safety and Efficacy of IV Ganaxolone as Adjuvant Therapy for Established Status Epilepticus

The study will enroll approximately 120 participants, at least 18 years of age with established (benzodiazepine-resistant) status epilepticus (ESE).

The primary objectives of the study are: (i) to determine an optimal IV ganaxolone dosing regimen in patients with ESE (benzodiazepine resistant) while receiving the initial second-line IV antiepileptic drug (AED) and (ii) to determine initial safety and efficacy of optimized IV ganaxolone regimen as an adjuvant to the initial second-line IV AED in patients with ESE

The secondary objectives of the study are to: (i) assess the time to SE cessation following IV ganaxolone administration, (ii) identify the proportion of participants requiring escalation of care to treat seizures within 24 hours of ganaxolone initiation, and (iii) to assess the time to escalation of care.

This study will be performed in emergency departments (ED) and the participants will be experiencing active convulsions or have electrographic evidence of SE despite having received benzodiazepines.

The study will be composed of 2 phases, the initial open-label, dose optimization phase and subsequent double blind placebo-controlled phase. During the open-label portion of the study, multiple sequential cohorts of participants will be assessed. Each participant in the first cohort may receive up to 3 sequential infusions of IV ganaxolone. The first infusion will always begin with a bolus of ganaxolone. The second and third infusions will not be initiated with a corresponding bolus if initiated within 30 minutes of prior infusion discontinuation. After the completion of each participant cohort and prior to initiation of the next participant cohort, the dosing regimen will be adjusted using a predetermined algorithm. The first administration of IV ganaxolone will begin with a 20-mg bolus administered over 3 minutes, followed by a 2-hour continuous IV infusion at 60 mg/hour. If clinical convulsions or electrographic SE recurs after the first infusion is completed but within 12 hours from the start of the first bolus of IV ganaxolone an optional 20 mg bolus followed by a 4-hour continuous infusion at 60 mg/hour may be administered. If convulsions or electrographic SE resumes after completion of the second infusion, an optional third 20-mg bolus and a 4-hour continuous infusion at 60 mg/hour may be administered. The need for the additional infusions will be assessed by the investigator only if the participant remains in the ED. The aggregate length of the continuous infusion in the first participant cohort will be 2 to 10 hours (2+4+4 hours).

A ganaxolone regimen selected during the dose optimization phase, consisting of an IV bolus followed by continuous infusion, will be used during the second, double-blind phase of the study.

The ganaxolone infusion will be stopped during either phase of the study if IV anesthetics (third line agents) are initiated to control clinical convulsions or electrographic SE despite ongoing ganaxolone administration. After ganaxolone has been discontinued, the follow-up period assessments/procedures will be collected for approximately 4 weeks.

After the completion of each participant cohort and prior to initiation of the next, the dosing regimen will be adjusted using a predetermined algorithm. The bolus dose will be adjusted based on the safety and efficacy observed during the first 30 minutes after the bolus initiation. The continuous infusion rate will be adjusted based on the safety and efficacy observed during the infusion. The duration of the IV infusion will be adjusted based on efficacy observed during 12 hours after the start of the first bolus and after completion of the infusion.

Efficacy will be defined as cessation of clinical convulsions and/or absence of electrographic evidence of SE on rapid EEG. The optimized dosing regimen selected in the first phase of the study, consisting of an IV bolus followed by infusion, will be used during the second phase, a double-blind comparison with placebo.

During both phases of the study, ganaxolone (IV ganaxolone in the dose optimization phase or IV ganaxolone versus placebo in the double-blind phase) will be initiated no earlier than 5 minutes after the last dose of benzodiazepine and concurrent (between 15 minutes before or 10 minutes after) with initiation of the first IV AED. The administration of this initial IV AED will take precedence over the administration of ganaxolone.

EEG will be required during the study. The initial qualifying SE episode will be defined clinically if the patient is actively experiencing convulsions but will require confirmation by EEG if possible and if ongoing seizures are not clinically obvious. Status epilepticus cessation will be assessed by the investigator based on the combination of clinical features and EEG data. Retrospective confirmatory analysis of EEG data will be performed by a central reader.

1. A method for treating established status epilepticus (ESE), comprising administering to a subject in need thereof a course of ganaxolone comprising:
a) an intravenous bolus of ganaxolone in an amount sufficient to suppress ESE; and
b) a continuous intravenous infusion of ganaxolone in an amount sufficient to prevent SE recurrence, wherein the continuous intravenous infusion i) is initiated periprocedural with the intravenous bolus, ii) is administered for a treatment period of about 2 hours.

2. The method of claim 1, wherein the continuous intravenous infusion is initiated immediately following the administration of the intravenous bolus.

3. The method of claim 1, wherein the intravenous bolus and the continuous intravenous infusion does not result in anesthesia of the subject.

4. The method of any one of claims 1-3, wherein the intravenous bolus produces a ganaxolone plasma concentration in the subject of at least about 400 ng/ml to about 1000 ng/ml.

5. The method of any one of claims 1-3, wherein the continuous intravenous infusion produces a ganaxolone plasma concentration in the subject of at least about 400 ng/ml to about 1000 ng/ml throughout the treatment period.

6. The method of the preceding claims wherein about 15 mg to about 25 mg of ganaxolone is administered to the subject at the initiation of the intravenous bolus.

7. The method of any one of the preceding claims, wherein about 20 mg of ganaxolone is administered to the subject at the initiation of the intravenous bolus.

8. The method of any one of the preceding claims, wherein the intravenous bolus is administered to the subject for about 1 minute to about 5 minutes.

9. The method of any one of any one of the preceding claims, wherein about 40 mg of ganaxolone per hour to about 80 mg of ganaxolone per hour are infused into the subject during the continuous infusion treatment period.

10. The method of claim 9, wherein about 60 mg of ganaxolone per hour is infused into the subject during the continuous infusion treatment period.

11. The method of any one of the preceding claims, further comprising administering to a subject in need thereof one or more additional courses of ganaxolone; wherein the one or more additional courses of ganaxolone comprises an intravenous bolus of ganaxolone and an intravenous continuous infusions of ganaxolone.

12. The method of claim 11, wherein a second course is administered to a subject that has seizure re-lapse within about 12 hours from the initiation of the intravenous bolus of ganaxolone; wherein the continuous intravenous infusion is administered for a treatment period of about 4 hours.

13. The method of claim 12, wherein the second course produces a ganaxolone plasma concentration in the subject of at least about 400 ng/ml throughout the treatment period.

14. The method of any one of claims 11-13, wherein a third course is administered to a subject that has seizure re-lapse within about 12 hours from the initiation of the second course; wherein the continuous intravenous infusion is administered for a treatment period of about 4 hours.

15. The method of claim 14, wherein the third course produces a ganaxolone plasma concentration in the subject of at least about 400 ng/ml throughout the treatment period.

16. The method of claim 12, wherein the intravenous continuous infusion is initiated concurrently with administration of the first additional intravenous bolus.

17. The method of claim 12 or 16, wherein about 15 mg to about 25 mg of ganaxolone is administered to the subject at the initiation of the one or more additional courses.

18. The method of claim 17, wherein about 20 mg of ganaxolone is administered to the subject at the initiation of the of the one or more additional courses.

19. The method of any one of claims 12 or 16-18, wherein the intravenous bolus is administered to the subject for about 1 minute to about 5 minutes.

20. The method of claim 12, wherein about 40 mg of ganaxolone per hour to about 80 mg of ganaxolone per hour are infused into the subject during the continuous intravenous infusion treatment period.

21. The method of claim 20, wherein about 60 mg of ganaxolone per hour is infused into the subject during the continuous infusion treatment period.

22. The method of any one of the preceding claims, wherein the method does not comprise a taper period.

23. The method of any one of the preceding claims, wherein the subject failed first-line seizure treatment.

24. The method of any one of claim 23, wherein the first-line seizure treatment is benzodiazepine.

25. The method of any one of the preceding claims, wherein the subject is monitored by electroencephalogram (EEG).

26. The method of any one of the preceding claims, wherein seizure activity in the subject is monitored by EEG.

27. The method of any of the preceding claims, wherein the intravenous administration of ganaxolone further comprises sulfobutylether-β-cyclodextrin.

28. The method of claim 27, wherein ganaxolone and sulfobutylether-β-cyclodextrin are administered in one formulation.

29. The method of any one of the preceding claims, wherein ganaxolone is administered as adjuvant to a second line IV AED.
